(19) United States
(12) Patent Application Publication
(10) Pub. No.: US 2017/0296487 A1
(43) Pub. Date: Oct. 19, 2017

(54) REDUCING REFLUX WHILE SLEEPING BY STIMULATING SALIVA WITH ADHERING TROCHES

(71) Applicant: OraHealth Corp., Bellevue, WA (US)

(72) Inventor: Jeffrey T. Haley, Mercer Island, WA (US)

(73) Assignee: OraHealth Corp., Bellevue, WA (US)

(21) Appl. No.: 15/638,367

(22) Filed: Jun. 30, 2017

Related U.S. Application Data
(63) Continuation of application No. 14/910,699, filed on Feb. 6, 2016, now abandoned.
(60) Provisional application No. 61/863,836, filed on Aug. 8, 2013.

(51) Int. Cl. A61K 31/047; A61K 31/19; A61K 9/20; A61K 9/00; A61K 36/28; A61J 3/06

(52) U.S. Cl. CPC A61K 31/047; A61K 36/28; A61K 31/19; A61J 3/06; A61K 9/205; A61K 9/0056; A61K 9/2086

(57) ABSTRACT

A method of reducing reflux from a stomach toward a mouth while sleeping in a person in need thereof by providing to the person an adhering, bilayer troche, comprising a first layer comprising an adhesive powder, and a second layer comprising a substrate that slowly dissolves or erodes in saliva and an ingredient to be released, wherein the ingredient stimulates saliva production in the mouth; and instructing the person to adhere one or more troches in their mouth before going to sleep, thereby causing frequent swallowing of stimulated saliva while sleeping resulting in reduced reflux from the stomach.

### BACKGROUND

Gastro-esophageal reflux disease (GERD), also called acid reflux, is caused by stomach acids rising into the esophagus. If untreated, it can cause an ulcer of the esophagus, a type of peptic ulcer, can cause cancer, and can erode teeth. GERD is typically worst while sleeping when the body is reclined so that gravity does not hold stomach acid down and swallowing is infrequent because saliva flow is lowest while sleeping. During the day, one can reduce acid reflux by stimulating saliva and therefore frequent swallowing through the use of chewing gum or slowly dissolving lozenges.

### SUMMARY OF THE INVENTION

In one aspect, a method is provided for reducing reflux from a stomach toward a mouth while sleeping in a person in need thereof by providing to the person an adhering, bilayer troche, the troche comprising, i. a first layer comprising an adhesive powder, and ii. a second layer comprising a substrate that slowly dissolves or erodes in saliva and an ingredient to be released, wherein the ingredient stimulates saliva production in the mouth; and b. instructing the person to adhere one or more troches in their mouth before going to sleep, thereby causing frequent swallowing of stimulated saliva while sleeping resulting in reduced reflux from the stomach.

In another aspect, a method is provided for reducing reflux from a stomach toward a mouth while sleeping comprising, adhering in the mouth before going to sleep a slowly dissolving, adhering, bilayer troche that dissolves while sleeping and releases an ingredient that stimulates saliva production while sleeping, thereby causing frequent swallowing of stimulated saliva resulting in reduced reflux from the stomach, wherein the troche comprises a first layer comprising an adhesive powder, and a second layer comprising a substrate that slowly dissolves or erodes in saliva and an ingredient to be released, wherein the ingredient stimulates saliva production in the mouth.

In another aspect, a kit is provided. The kit comprises, a bilayer, adhering troche, wherein the troche comprises a first layer comprising an adhesive powder, and a second layer comprising a substrate that slowly dissolves or erodes in saliva and an ingredient to be released, wherein the ingredient stimulates saliva production in the mouth thereby causing frequent swallowing of stimulated saliva resulting in reduced reflux from the stomach; and instructions to adhere one or more of the troches in the mouth before sleeping.

In embodiments, the troche may be from about 5 mm to about 18 mm in each of two dimensions. In embodiments, the ingredient that stimulates saliva production comprises flavor molecules. Flavor molecules may comprise a sweet polyol, including xylitol, sorbitol, maltitol, erythritol and mannitol, or a synthetic sweetener, including sucralose, neotame, aspartame, acesulfame potassium and saccharin, or stevia.

In embodiments, the person is instructed to adhere one or more troches to an outside of a molar or gum adjoining the outside of the molar.

### BRIEF DESCRIPTION OF THE DRAWING

FIG. 1 shows a side view of a bi-layer adhering troche made with a tablet press.

### DETAILED DESCRIPTION

A troche with a slowly dissolving substrate and an ingredient that stimulates saliva may be made by mixing dry granular powders. The substrate comprises at least one powder that will slowly dissolve or erode in saliva, such as a carbohydrate like inulin, r polydextrose or a polyol.

The ingredient may be flavor molecules that comprise any combination of sweet, sour, salty, bitter or savory. All of these flavors will stimulate saliva production. Typically, flavors are sweet, savory and salty.

If a sweet flavor is used, it is desirable to not use a cariogenic carbohydrate like sugar because these carbohydrates promote tooth decay. Some suitable non-cariogenic carbohydrates include polyols, e.g., xylitol, sorbitol, maltitol, erythritol, and mannitol. Stevia and the synthetic sweeteners such as sucralose, neotame, aspartame, acesulfame potassium, and saccharin are also suitable.

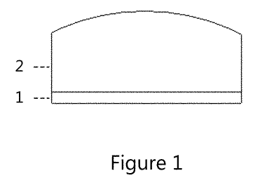

The troches may be formed by pressing powders into a tablet as shown in FIG. 1 by using a bi-layer tablet press. Troches will generally be at least 5 mm diameter if round, and at least 5 mm in each of two dimensions if not round. Generally, troches will be from 5-18 mm diameter or 5-18 mm in each of two dimensions. Grain sizes of 50 to 350 microns are typical. The grains may be granulated with a coating of binder gum on the outside, such as

Danisco Xylitab® 200 which is granulated with up to 2% carboxymethylcellulose (CMC—cellulose gum) as a compression binder. Alternatively, grains of material not coated with a gum, such as Danisco Xylitab® 300, may be mixed with a binder gum powder such as CMC and then pressed. Some of the polyols, such as sorbitol and maltitol, compress well and may be used without a compression binder.

The adhesive molecules may comprise one or more of acacia gum, gelatin, alginate, starch, pectin, polyvinylpyrolidone, carboxymethylcellulose, hydroxymethylcellulose, polyvinyl acid, polyacrylic acid, and carbopol. Acacia gum adheres very well to teeth and gingiva, and it does not dissolve too fast or leave an unattractive mouth feel. On the surface designed to be adherent, between 80% and 100% acacia gum is preferred for good adhesion. Acacia gum may be mixed with an alkalizer to obtain and keep it pH neutral. A suitable ratio for pH neutrality is 1 unit calcium carbonate (CaCO3) to 30 units acacia gum. Alternatively, the adhesive molecules may comprise one or more of gelatin, alginate, starch, pectin, polyvinylpyrolidone, carboxymethylcellulose, hydroxymethylcellulose, polyvinyl acid, polyacrylic acid, and carbopol. Percentages of these molecules that exhibit adherent function are well known.

The adherent layer can be quite thin. In tests on a troche of about 12 mm in diameter by 4 to 5 mm thick, a suitable thickness of a layer of about 97% acacia gum was about 0.5-about 0.9 mm.

An adhering troche held in a human mouth erodes, releasing flavor molecules over time, allowing delivery of saliva-stimulating ingredients without the effect on appearance of chewing or having a candy in one's mouth. Moreover, an adhering troche can be used safely while sleeping. While awake, a troche comprising 0.7 g xylitol and 4% CMC and about 4.5 mm thick dissolves in a human mouth with normal saliva flow in 30-70 minutes, although the time depends at least in part on saliva flow, saliva chemistry, and mouth movement. While asleep, a troche having the same composition lasts generally at least about 2 hours and as long as about 8 hours.

An exemplary method for making bi-layer troches using a typical press, comprises placing the ingredient-releasing powder in the die, sitting on the lower punch, tamping the powder with the upper punch, which leaves the surface having the shape of the upper punch face, adding powder of the adhesive layer, and pressing with an upper punch. The shape of the upper punch that presses the ingredient-releasing powder and that presses the adhesive powder may be the same or different. Thus, a troche may have an adhesive side and an ingredient side with different shapes. For example, the ingredient-releasing powder may be tamped with a flat or essentially flat or rounded punch and the adhesive powder tamped with a flat or essentially flat punch or a dished (e.g., dimpled) punch. In another example, a troche has a dimpled adhesive face and a rounded ingredient face. An example of a bi-layer tablet is the adhering xylitol troche disclosed in U.S. patent application Ser. No. 11/800,381, filed May 4, 2007, which is incorporated in its entirety.

Bilayer, adhering troches may be supplied in a kit with instructions for use. For use while sleeping, the troche is best not adhered to the roof of the mouth for safety reasons, because the person might pry it loose with their tongue while sleeping in which case it could fall into the airway. Instead, typically, it is adhered to the outside of a molar or adjoining gums. One or more troches, e.g., two, three, four, five or more, may be used during sleeping. The number of troches that can be used may be limited by the size of the mouth.

To verify that the described method of reducing reflux while sleeping works, the inventor supplied adhering troches as described above to a person who usually tastes the sourness of stomach acid in their mouth during the last 1-2 hours of sleep before waking. The person adhered two troches in their mouth each night at bedtime, one on each side of the mouth. Each troche released 500 mg of xylitol and no other flavor. During each night of sleep when the troches were used, the person reported no sour taste in his mouth during the night. When the person stopped using the troches for a night, he again reported tasting the sour stomach acid during the last hour of sleep before arising.

While particular embodiments of the invention have been described above the scope of the invention should not be limited by the above descriptions but rather limited only by the following claims.

1. A method of reducing reflux from a stomach toward a mouth while sleeping in a person in need thereof comprising:
a. providing to the person an adhering, bilayer troche, the troche comprising:
i. a first layer comprising an adhesive that adheres in a human mouth, and
ii. a second layer comprising a substrate that slowly dissolves or erodes in saliva and an ingredient to be released, wherein the ingredient stimulates saliva production in the mouth; and
b. instructing the person to adhere in their mouth before going to sleep one or more of the troches, thereby causing frequent swallowing of stimulated saliva while sleeping resulting in reduced reflux from the stomach.

2. The method of claim 1 where the ingredient that stimulates saliva production comprises flavor molecules.

3. The method of claim 2 wherein the flavor molecules comprise a sweet polyol.

4. The method of claim 3 wherein the sweet polyol molecules are selected from the group consisting of xylitol, sorbitol, maltitol, erythritol and mannitol.

5. The method of claim 2, wherein the flavor molecules comprise a synthetic sweetener.

6. The method of claim 5, wherein the synthetic sweetener molecules are selected from the group consisting of sucralose, neotame, aspartame, acesulfame potassium and saccharin.

7. The method of claim 2, wherein the flavor molecules comprise stevia.

8. The method of claim 1, wherein the troche is from about 5 mm to about 18 mm in each of two dimensions.

9. The method of claim 1, wherein the person is instructed to adhere the troche to an outside of a molar or gum adjoining the outside of the molar.

10. A method of reducing reflux from a stomach toward a mouth while sleeping comprising:
adhering in the mouth before going to sleep a slowly dissolving, adhering, bilayer troche that dissolves while sleeping and releases an ingredient that stimulates saliva production while sleeping, thereby causing frequent swallowing of stimulated saliva resulting in reduced reflux from the stomach,
wherein the troche comprises
i. a first layer comprising an adhesive that adheres in a human mouth, and
ii. a second layer comprising a substrate that slowly dissolves or erodes in saliva and an ingredient to be released, wherein the ingredient stimulates saliva production in the mouth.

11. The method of claim 10, wherein the ingredient comprise flavor molecules.

12. The method of claim 11, wherein the flavor molecules comprise a sweet polyol.

13. The method of claim 12, wherein the sweet polyol molecules are selected from the group consisting of xylitol, sorbitol, maltitol, erythritol and mannitol.

14. The method of claim 11, wherein the flavor molecules comprise a synthetic sweetener.

15. The method of claim 14, wherein the synthetic sweetener molecules are selected from the group consisting of sucralose, neotame, aspartame, acesulfame potassium and saccharin.

16. The method of claim 11, wherein the flavor molecules comprise stevia.

17. The method of claim 10, wherein the troche is from about 5 mm to about 18 mm in each of two dimensions.

18. The method of claim 10, wherein the adhering troche is adhered to an outside of a molar or gums adjoining the outside of the molar.

19. A kit, comprising:
(a) a bilayer, adhering troche, wherein the troche comprises
i. a first layer comprising an adhesive that adheres in a human mouth, and
ii. a second layer comprising a substrate that slowly dissolves or erodes in saliva and an ingredient to be released, wherein the ingredient stimulates saliva production in the mouth thereby causing frequent swallowing of stimulated saliva resulting in reduced reflux from the stomach; and
(b) instructions to adhere one or more of the troches in the mouth before sleeping.

20. (canceled)

21. (canceled)

22. The kit of claim 19, wherein the ingredient comprises flavor molecules.

23. The kit of claim 22, wherein the flavor molecules comprise a sweet polyol selected from the group consisting of xylitol, sorbitol, maltitol, erythritol and mannitol.

24. The kit of claim 22, wherein the flavor molecules comprise a synthetic sweetener selected from the group consisting of sucralose, neotame, aspartame, acesulfame potassium and saccharin.

25. The kit of claim 22, wherein the flavor molecules comprise stevia.
